(19) United States
(12) Patent Application Publication
(10) Pub. No.: US 2007/0155972 A1
(43) Pub. Date: Jul. 5, 2007

(54) Method for separating trioxane from a mixture containing trioxane, formaldehyde and water

(75) Inventors: Neven Lang, Mannheim (DE); Eckhard Stroefer, Mannheim (DE); Achim Stammer, Freinsheim (DE); Thorsten Friese, Mannheim (DE); Markus Siegert, Heidelberg (DE); Michael Ott, Neckargemund (DE); Hans Hasse, Kaiserslautern (DE); Thomas Grutzner, Stuttgart (DE); Sergej Blagov, Stuttgart (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 10/583,696

(22) PCT Filed: Dec. 21, 2004
(86) PCT No.: PCT/EP04/14531

§ 371 Date: Jun. 20, 2006

(30) Foreign Application Priority Data
Dec. 23, 2003 (DE) 103 61 518.0

(51) Int. Cl. C07D 305/12; C07C 41/03

(52) U.S. Cl. 549/326; 568/679; 564/396

(57) ABSTRACT

A process for removing trioxane from a mixture I of formaldehyde, trioxane and water, by a) distilling the mixture I in a first distillation stage at a pressure of from 0.1 to 2 bar to obtain a stream II which comprises formaldehyde and a stream III which comprises predominantly trioxane and additionally water and formaldehyde, b) mixing the stream III with a recycle stream VII which comprises predominantly trioxane and additionally water and formaldehyde to obtain a stream IIIa which comprises predominantly trioxane and additionally water and formaldehyde, c) distilling the stream IIIa, if appropriate after removing low boilers from the stream III or IIIa in a further distillation stage, in a second distillation stage at a pressure of from 0.2 to 10 bar, the pressure in the second distillation stage being at least 0.1 bar higher than the pressure in the first distillation stage, to obtain a stream IV of trioxane and a stream V which comprises predominantly trioxane and additionally water and formaldehyde, d) distilling the stream V in a third distillation stage at a pressure of from 0.1 to 4 bar to obtain a stream VI which comprises predominantly water and additionally formaldehyde, and the recycle stream VII which comprises predominantly trioxane and additionally water and formaldehyde, e) if appropriate, distilling the stream VI in a fourth distillation stage to obtain a stream VIII which comprises predominantly water, and a stream IX which comprises predominantly formaldehyde.

The invention relates to a process for removing trioxane from a trioxane/formaldehyde/water mixture, and also to a process for preparing trixane.

Trioxane is generally prepared by distilling aqueous formaldehyde solution in the presence of acidic catalysts. The trioxane is subsequently removed from the distillate comprising formaldehyde and water by extraction with halogenated hydrocarbons such as methylene chloride or 1,2-dichloroethane, or other, water-immiscible solvents.

DE-A 1 668 867 describes a process for removing trioxane from mixtures comprising water, formaldehyde and trioxane by extraction with an organic solvent. In this process, an extraction section consisting of two subsections is charged at one end with a customary organic, virtually water-immiscible extractant for trioxane, and at the other end with water. Between the two subsections, the distillate of the trioxane synthesis to be separated is fed. On the side of the solvent feed, an aqueous formaldehyde solution is then obtained, and on the side of the water feed, a virtually formaldehyde-free solution of trioxane in the solvent. In one example, the distillate which is obtained in the trioxane synthesis and is composed of 40% by weight of water, 35% by weight of trioxane and 25% by weight of formaldehyde is metered into the middle section of a pulsation column, and methylene chloride is fed at the upper end of the column and water at the lower end of the column. In this case, an about 25% by weight solution of trioxane in methylene chloride is obtained at the lower end of the column and an about 30% by weight aqueous formaldehyde solution at the upper end of the column.

A disadvantage of this procedure is the occurrence of extractant which has to be purified. Some of the extractants used are hazardous substances (T or T⁺ substances in the context of the German Hazardous Substances Directive), whose handling entails special precautions.

DE-A 197 32 291 describes a process for removing trioxane from an aqueous mixture which consists substantially of trioxane, water and formaldehyde, by removing trioxane from the mixture by pervaporation and separating the trioxane-enriched permeate by rectification into trioxane and an azeotropic mixture of trioxane, water and formaldehyde. In the example, an aqueous mixture consisting of 40% by weight of trioxane, 40% by weight of water and 20% by weight of formaldehyde is separated in a first distillation column under atmospheric pressure into a water/formaldehyde mixture and into an azeotropic trioxane/water/formaldehyde mixture. The azeotropic mixture is passed into a pervaporation unit which contains a membrane composed of polydimethylsiloxane with a hydrophobic zeolite. The trioxane-enriched mixture is separated in a second distillation column under atmospheric pressure into trioxane and, in turn, into an azeotropic mixture of trioxane, water and formaldehyde. This azeotropic mixture is recycled before the pervaporation stage.

A disadvantage of this procedure is the very high capital costs for the pervaporation unit.

It is an object of the invention to provide a process for removing trioxane from azeotropic trioxane/formaldehyde/water mixtures, which does not need any of the extraction steps or pervaporation steps of the prior art.

This object is achieved by a process for removing trioxane from a mixture I of formaldehyde, trioxane and water, by
a) distilling the mixture I in a first distillation stage at a pressure of from 0.1 to 2 bar to obtain a stream II which comprises formaldehyde and a stream III which comprises predominantly trioxane and additionally water and formaldehyde,
b) mixing the stream III with a recycle stream VII which comprises predominantly trioxane and additionally water and formaldehyde to obtain a stream IIIa which comprises predominantly trioxane and additionally water and formaldehyde,
c) distilling the stream IIIa, if appropriate after removing low boilers from the stream III or IIIa in a further distillation stage, in a second distillation stage at a pressure of from 0.2 to 10 bar, the pressure in the second distillation stage being at least 0.1 bar higher than the pressure in the first distillation stage, to obtain a stream IV of trioxane and a stream V which comprises predominantly trioxane and additionally water and formaldehyde,
d) distilling the stream V in a third distillation stage at a pressure of from 0.1 to 4 bar to obtain a stream VI which comprises predominantly water and additionally formaldehyde, and the recycle stream VII which comprises predominantly trioxane and additionally water and formaldehyde,
e) if appropriate, distilling the stream VI in a fourth distillation stage to obtain a stream VIII which comprises predominantly water, and a stream IX which comprises predominantly formaldehyde.

The mixtures comprise a component “predominantly” when the component in question constitutes the main component, i.e. the component having the larger or largest proportion by mass. The proportion by mass of the predominant component in the mixture is preferably at least 50% by weight.

It is known that trioxane, formaldehyde and water form a ternary azeotrope which, at a pressure of 1 bar, has the composition of 69.5% by weight of trioxane, 5.4% by weight of formaldehyde and 25.1% by weight of water.

According to the invention, this azeotrope is circumvented by pressure swing distillation, in which a first and a second distillation are carried out at different pressures. In a first distillation column which is operated at lower pressure, the starting mixture is separated into a trioxane/water mixture having low formaldehyde content and a substantially trioxane-free formaldehyde/water mixture. The trioxane-free formaldehyde/water mixture may be recycled into the trioxane synthesis. In a further distillation column operated at higher pressure, the trioxane/formaldehyde/water mixture is separated into pure trioxane and a trioxane/formaldehyde/water mixture having a low trioxane content.

Suitable distillation columns are any distillation columns such as packed or tray columns. The columns may contain any internals, structured packings or random packings.

The pressure in the second distillation stage is at least 0.1 bar higher than the pressure in the first distillation stage. In general, this pressure differential is from 0.5 to 10 bar, preferably from 1 to 5 bar.

All pressure data relate to the pressure at the top of the column in question.

The first distillation stage is carried out at a pressure of from 0.1 to 2 bar, preferably from 0.5 to 2 bar, for example 1 bar. The first distillation stage is generally carried out in a distillation column having at least 2, preferably from 2 to 50, theoretical plates. In general, the stripping section includes at least 25% of the number of theoretical plates of the column. The stripping section preferably includes from 50 to 90% of the theoretical plates of the column. The mixture I, preferably a feed stream I which is obtained in a preceding trioxane synthesis, generally contains from 35 to 80% by weight of formaldehyde, from 25 to 45% by weight of water and from 1 to 30% by weight of trioxane. This mixture I is separated into a stream II which is preferably removed at the bottom of the column, and a stream III which is preferably removed at the top of the column. The stream II generally contains from 51 to 80% by weight of formaldehyde, from 20 to 49% by weight of water and from 0 to 1% by weight of trioxane. The stream III generally contains from 1 to 15% by weight of formaldehyde, from 15 to 35% by weight of water and from 60 to 80% by weight of trioxane. The stream II is preferably recycled into the trioxane synthesis.

The mixture I which is distilled in the first distillation column may also be obtained by reactive distillation in the first distillation column (which is then designed as the reaction column) (see below). In this case, the formaldehydic bottom draw stream II may be small and serve merely to discharge high boilers. Alternatively, the bottom draw stream II may be at least partly recycled into the reaction column.

The stream III is combined with a recycle stream VII which is obtained in the third distillation stage (see below) to give stream IIIa. The stream IIIa generally contains from 3 to 20% by weight of formaldehyde, from 10 to 30% by weight of water and from 60 to 80% by weight of trioxane.

The streams I, III, IIIa, V and VII may also contain up to 15% by weight of low boilers. Typical low boilers which can be formed in the trioxane synthesis and the subsequent distillative separation are methyl formate, methylal, dimethoxydimethyl ether, trimethoxydimethyl ether, methanol, formic acid, and also further hemiacetals and full acetals. To remove these low boilers, a further distillation stage (low boiler removal stage) may optionally be carried out between the first and the second distillation stage. In this case, the low boilers are preferably removed via the top of a low boiler removal column which is preferably operated at a pressure of from 1 to 2 bar. In general, the low boiler removal column has at least 5 theoretical plates, preferably from 15 to 50 theoretical plates. The stripping section of this column preferably includes from 25 to 90% of the theoretical plates of this column. Preference is given to carrying out this low boiler removal. It is also possible to remove the low boilers from the stream III and subsequently to combine the stream III with the recycle stream VII to give the stream IIIa.

When a low boiler removal is dispensed with, the low boilers are obtained with the trioxane stream IV. This then results in trioxane of lower purity.

The stream IIIa is separated in a second distillation stage at a pressure of from 0.2 to 8 bar into a stream IV composed of trioxane and a stream V which comprises predominantly trioxane and additionally water and formaldehyde. This second distillation stage is carried out at a pressure of from 0.2 to 10 bar, preferably from 2.5 to 8 bar, for example at 4 bar. In general, this second distillation stage is carried out in a distillation column having at least 2 theoretical plates, preferably from 5 to 50 theoretical plates, and the stream IV is obtained as a bottom draw stream or as a side draw stream in the stripping section of the column, and the stream V is obtained as a top draw stream. In general, the stripping section of the distillation column includes from 50 to 90% of the theoretical plates of this column.

In general, the stream IV contains from 95 to 100% by weight, preferably from 99 to 100% by weight, of trioxane, and from 0 to 5% by weight, preferably from 0 to 1% by weight, of water and secondary components. Secondary components are in particular the abovementioned low boilers, but also components having a higher boiling point than trioxane. The content of water and secondary components in the trioxane stream IV is more preferably <0.1%. It may even be <0.01%. The stream V generally contains from 5 to 20% by weight of formaldehyde, from 15 to 35% by weight of water and from 50 to 80% by weight of trioxane.

The stream V is separated in a third distillation stage at a pressure of from 0.1 to 4 bar into a stream VI which comprises predominantly water and additionally formaldehyde, and the recycle stream VII which comprises predominantly trioxane and additionally water and formaldehyde. Preference is given to carrying out the third distillation stage at a pressure of from 0.1 to 1 bar, for example 0.2 bar. In general, the third distillation stage is carried out in a distillation column having at least one theoretical plate, preferably from 2 to 20 theoretical plates, and the stream VI is obtained as a bottom draw stream and the steam VII as a top draw stream. The stripping section of this column preferably includes from 40 to 90% of the theoretical plates of this column.

The stream VI generally contains from 10 to 25% by weight of formaldehyde, from 75 to 90% by weight of water and from 0 to 1% by weight of trioxane. The stream VII generally contains from 5 to 20% by weight of formaldehyde, from 10 to 30% by weight of water and from 60 to 80% by weight of trioxane.

The present invention also provides a process for preparing trioxane from an aqueous formaldehyde solution, by preparing the use stream I comprising formaldehyde, trioxane and water from an aqueous formaldehyde solution in a preceding trioxane synthesis stage and subsequently removing trioxane from the stream I as described above. Alternatively, the trioxane synthesis and the first distillation stage may be combined in a reactive distillation.

In one embodiment of the process according to the invention, a stream X composed of an aqueous formaldehyde solution of a preceding trioxane synthesis stage is fed and converted in the presence of acidic homogeneous or heterogeneous catalysts such as ion exchange resins, zeolites, sulfuric acid and p-toluenesulfonic acid at a temperature of generally from 70 to 130° C. Operation may be effected in a distillation column or an evaporator (reactive evaporator). The product mixture of trioxane/formaldehyde and water is then obtained as a vaporous vapor draw stream of the evaporator or as a top draw stream at the top of the column. The trioxane synthesis stage may also be carried out in a fixed bed or fluidized bed reactor over a heterogeneous catalyst, for example an ion exchange resin or zeolite.

In a further embodiment of the process according to the invention, the trioxane synthesis stage and the first distillation stage are carried out as a reactive distillation in one reaction column. This may contain a fixed catalyst bed of a heterogeneous acidic catalyst in the stripping section. Alternatively, the reactive distillation may also be carried out in the presence of a homogeneous catalyst, in which case the acidic catalyst is present in the column bottom together with the aqueous formaldehyde solution.

In general, the aqueous formaldehyde solution which is fed to the trioxane synthesis stage contains from 55 to 85% by weight of formaldehyde and from 15 to 45% by weight of water. This solution may be obtained in a preceding concentration step from an aqueous formaldehyde solution having low formaldehyde concentration. The concentration step may be carried out, for example, in an evaporator, preferably a falling-film evaporator.

The preceding concentration step may be carried out, for example, as described in DE-A 199 25 870.

The resulting pure trioxane, whose purity may be >99% by weight, >99.9% by weight or even >99.99% by weight, is preferably used to prepare polyoxymethylene (POM), polyoxymethylene derivatives such as polyoxymethylene dimethyl ether (POMDME) and diaminodiphenylmethane (MDA).

The invention is illustrated in detail hereinbelow with reference to the drawing.

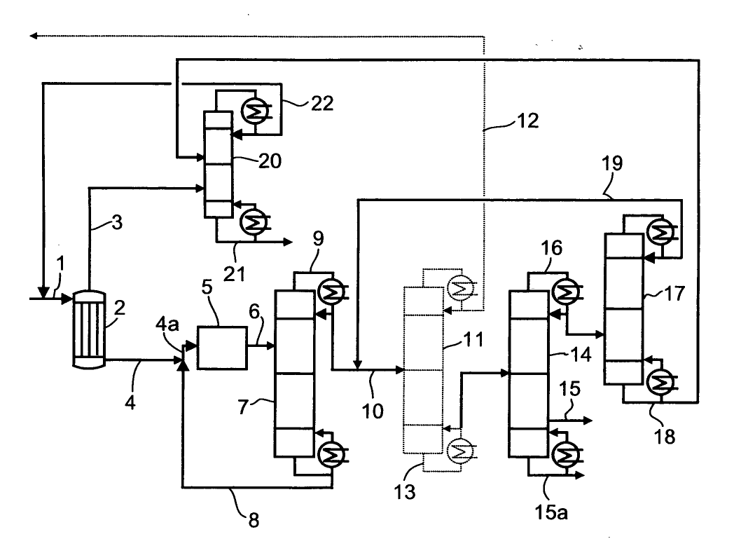

FIG. 1 shows an example of an embodiment of the process according to the invention.

An aqueous formaldehyde 1 having a formaldehyde content of typically from 50 to 65% by weight is fed to the evaporator 2, for example a thin-film evaporator, falling-film evaporator or helical-tube evaporator. The vapor draw stream 3 of the evaporator which is obtained is a formaldehyde-depleted aqueous solution, the bottom draw stream 4 of the evaporator a formaldehyde-rich aqueous solution having a formaldehyde content of typically from 55 to 80% by weight. This is fed to the trioxane synthesis reactor 5 which is configured as an evaporator, stirred tank or fixed bed or fluidized bed reactor. The trioxane/formaldehyde/water mixture 6 leaving the trioxane synthesis reactor is fed to the first distillation column 7 and separated there into a formaldehyde/water stream 8 (stream II) and a formaldehyde/water/trioxane stream 9 (stream II). The stream 8 is obtained as a bottom draw stream and the stream 9 as a top draw stream. Stream 8 is combined with stream 4 and recycled as stream 4a into the reactor 5. Stream 9 is combined with the recycle stream 19 (stream VII) composed of formaldehyde/water and trioxane to give the stream 10 (stream IIIa). In a low boiler removal column 11, low boilers including methyl formate, methylal, dimethoxydimethyl ether and methanol may be removed overhead from the stream 10 as a stream 12. The bottom draw stream 13 is fed to the distillation column 14 and separated there into a stream 15 (stream IV) composed of substantially pure trioxane and a stream 16 (stream V) which comprises predominantly trioxane and additionally water and formaldehyde. Stream 15 may be obtained as a side draw stream in the stripping section of the column, preferably in gaseous form in the vicinity of the column bottom. In this case, the trioxane has particularly high purity. The bottom draw stream obtained may be a stream 15a which is enriched with high boilers such as tetraoxane and further high-boiling secondary components. The trioxane stream 15 may also be obtained as a bottom draw stream.

The stream 16 is fed to a third distillation column 17 and separated there into a stream 18 (stream VI) which comprises predominantly water and additionally formaldehyde, and the recycle stream 19 (stream VII) which comprises predominantly trioxane and additionally water and formaldehyde. The stream 18 is fed to a further distillation column 20 and separated there into a stream 21 consisting substantially of water and a stream 22 composed of formaldehyde-enriched aqueous formaldehyde solution. The vapor draw stream 3 of the evaporator 2 may also be fed into the column 20 to concentrate the formaldehyde contained therein. The formaldehyde/water stream 22 is recycled into the evaporator together with the feed stream 1.

### EXAMPLES

In the simulation of the process illustrated in the figure, streams 1, 4a, 6, 8, 9, 10, 15, 16, 18 and 19 of the compositions reported in the tables were obtained. The following parameters were assumed: the first distillation stage is carried out at a pressure of 1 bar in a column 7 having 16 theoretical plates. The reflux ratio is 1.8, the top temperature 91° C. and the bottom temperature 103° C. The feed 6 is disposed at the height of the 4th theoretical plate. The second distillation stage is carried out at a pressure of 4 bar in a column 14 having 8 theoretical plates. The reflux ratio is 1, the top temperature 133° C., and the temperature at the side draw 15, which is mounted at the height of the first theoretical plate, 165° C. The feed 13 is disposed at the height of the 5th theoretical plate. The third distillation stage is carried out at 0.2 bar in a column 17 having 5 theoretical plates. The reflux ratio is 0.7, the top temperature 51° C. and the bottom temperature 62° C. The feed 16 is disposed at the height of the 3rd theoretical plate. The fourth distillation stage is carried out at a pressure of 4 bar.

### Example 1

| | Stream | | | | | | | | | |
|---|---|---|---|---|---|---|---|---|---|---|
| | | 4a | 6 | 8 | 9 | 10 | 15 | 16 | 18 | 19 |
| | 1 | (X) | (I) | (II) | (III) | (IIIa) | (IV) | (V) | (VI) | (VII) |
| Mass flow | 1408 | 4361 | 4361 | 2952 | 1408 | 8148 | 1000 | 7148 | 408 | 6739 |
| rate [kg/h] | | | | | | | | | | |
| Formaldehyde | 0.76 | 0.71 | 0.48 | 0.69 | 0.05 | 0.11 | 0.00 | 0.13 | 0.17 | 0.13 |
| [% by wt] | | | | | | | | | | |
| Water | 0.24 | 0.29 | 0.29 | 0.31 | 0.24 | 0.19 | 0.00 | 0.21 | 0.83 | 0.17 |
| [% by wt.] | | | | | | | | | | |
| Trioxane | 0.00 | 0.00 | 0.23 | 0.00 | 0.71 | 0.70 | 1.00 | 0.66 | 0.00 | 0.70 |
| [% by wt.] | | | | | | | | | | |

### Example 2

| | Stream | | | | | | | | | |
|---|---|---|---|---|---|---|---|---|---|---|
| | | 4a | 6 | 8 | 9 | 10 | 15 | 16 | 18 | 19 |
| | 1 | (X) | (I) | (II) | (III) | (IIIa) | (IV) | (V) | (VI) | (VII) |
| Mass flow | 1456 | 5041 | 5041 | 2952 | 1408 | 8148 | 1000 | 4494 | 456 | 6739 |
| rate [kg/h] | | | | | | | | | | |
| Formaldehyde | 0.74 | 0.65 | 0.45 | 0.61 | 0.05 | 0.12 | 0.00 | 0.15 | 0.16 | 0.15 |
| [% by wt] | | | | | | | | | | |
| Water | 0.26 | 0.35 | 0.35 | 0.39 | 0.26 | 0.18 | 0.00 | 0.22 | 0.83 | 0.15 |
| [% by wt.] | | | | | | | | | | |
| Trioxane | 0.00 | 0.00 | 0.20 | 0.00 | 0.69 | 0.70 | 1.00 | 0.63 | 0.01 | 0.70 |
| [% by wt.] | | | | | | | | | | |

### Example 3

| | Stream | | | | | | | | | |
|---|---|---|---|---|---|---|---|---|---|---|
| | | 4a | 6 | 8 | 9 | 10 | 15 | 16 | 18 | 19 |
| | 1 | (X) | (I) | (II) | (III) | (IIIa) | (IV) | (V) | (VI) | (VII) |
| Mass flow | 1493 | 6280 | 6280 | 4787 | 1493 | 5785 | 1000 | 4785 | 493 | 4292 |
| rate [kg/h] | | | | | | | | | | |
| Formaldehyde | 0.77 | 0.60 | 0.44 | 0.55 | 0.10 | 0.10 | 0.00 | 0.12 | 0.30 | 0.10 |
| [% by wt] | | | | | | | | | | |
| Water | 0.23 | 0.40 | 0.40 | 0.45 | 0.23 | 0.22 | 0.00 | 0.27 | 0.70 | 0.22 |
| [% by wt.] | | | | | | | | | | |
| Trioxane | 0.00 | 0.00 | 0.16 | 0.00 | 0.67 | 0.68 | 1.00 | 0.61 | 0.00 | 0.68 |
| [% by wt.] | | | | | | | | | | |

### Example 4

| | Stream | | | | | | | | | |
|---|---|---|---|---|---|---|---|---|---|---|
| | | 4a | 6 | 8 | 9 | 10 | 15 | 16 | 18 | 19 |
| | 1 | (X) | (I) | (II) | (III) | (IIIa) | (IV) | (V) | (VI) | (VII) |
| Mass flow | 1449 | 5579 | 5579 | 4129 | 1449 | 4594 | 1000 | 3594 | 449 | 3145 |
| rate [kg/h] | | | | | | | | | | |
| Formaldehyde | 0.71 | 0.63 | 0.45 | 0.60 | 0.02 | 0.08 | 0.00 | 0.10 | 0.06 | 0.11 |
| [% by wt] | | | | | | | | | | |
| Water | 0.29 | 0.37 | 0.37 | 0.40 | 0.29 | 0.21 | 0.00 | 0.27 | 0.94 | 0.17 |
| [% by wt.] | | | | | | | | | | |
| Trioxane | 0.00 | 0.00 | 0.18 | 0.00 | 0.69 | 0.71 | 1.00 | 0.63 | 0.00 | 0.72 |
| [% by wt.] | | | | | | | | | | |

1-13. (canceled)

14. A process for removing trioxane from a mixture I of formaldehyde, trioxane and water, by
a) distilling the mixture I in a first distillation stage at a pressure of from 0.1 to 2 bar to obtain a stream II which comprises formaldehyde and a stream III which comprises predominantly trixoane and additionally water and formaldehyde,
b) mixing the stream III with a recycle stream VII which comprises predominantly trioxane and additionally water and formaldehyde to obtain a stream IIIa which comprises predominantly trioxane and additionally water and formaldehyde,
c) distilling the stream IIIa, if appropriate after removing low boilers from the stream III or IIIa in a further distillation stage, in a second distillation stage at a pressure of from 0.2 to 10 bar, the pressure in the second distillation stage being at least 0.1 bar higher than the pressure in the first distillation stage, to obtain a stream IV of trioxane and a stream V which comprises predominantly trioxane and additionally water and formaldehyde,
d) distilling the stream V in a third distillation stage at a pressure of from 0.1 to 4 bar to obtain a stream VI which comprises predominantly water and additionally formaldehyde, and the recycle stream VII which comprises predominantly trioxane and additionally water and formaldehyde,
e) if appropriate, distilling the stream VI in a fourth distillation stage to obtain a stream VIII which comprises predominantly water, and a stream IX which comprises predominantly formaldehyde.

15. The process according to claim 14, wherein the pressure in the second distillation stage is from 0.5 to 10 bar higher than the pressure in the first distillation stage.

16. The process according to claim 14, wherein the first distillation stage is carried out at a pressure of from 0.5 to 2 bar and the second distillation stage at a pressure of from 2.5 to8bar.

17. The process according to claim 14, wherein the third distillation stage is carried out at a pressure of from 0.1 to 1 bar.

18. The process according to claim 14, wherein the first distillation stage is carried out in a distillation column having at least two theoretical plates, the second distillation stage in a distillation column having at least 2 theoretical plates and the third distillation stage in a distillation column having at least one theoretical plate.

19. The process according to claim 14, characterized by the following composition of streams I-VII:
stream I: from 35 to 80% by weight of formaldehyde, from 25 to 45% by weight of water, from 1 to 30% by weight of trioxane;
stream II: from 51 to 80% by weight of formaldehyde, 20 to 49% by weight of water, 0 to 1% by weight of trioxane;
stream III: from 1 to 15% by weight of formaldehyde, 15 to 35% by weight of water, 60 to 80% by weight of trioxane;
stream IIIa: from 3 to 20% by weight of formaldehyde, 10 to 30% by weight of water, 60 to 80% by weight of trioxane;
stream IV: from 95 to 100% by weight of trioxane, 0 to 5% by weight of water and secondary components;
stream V: from 5 to 20% by weight of formaldehyde, 15 to 35% by weight of water, 50 to 80% by weight of trioxane;
stream VI: from 10 to 25% by weight of formaldehyde, 75 to 90% by weight of water, 0 to 1% by weight of trioxane;
stream VII: from 5 to 20% by weight of formaldehyde, 10 to 30% by weight of water, 60 to 80% by weight of trioxane,
and the streams I, III, IIIa, V and VII may also contain up to 15% by weight of low boilers selected from the group consisting of methyl formate, methylal, dimethoxydimethyl ether, trimethoxydimethyl ether, methanol, formic acid, and also further hemiacetals and full acetals.

20. The process according to claim 14, wherein a low boiler removal is undertaken in a further distillation stage between the first and the second distillation stage.

21. A process for preparing trioxane from an aqueous formaldehyde solution, by feeding a stream X of an aqueous formaldehyde of a trioxane synthesis stage and converting it under acidic conditions to obtain the mixture I, and removing trioxane from the stream I by the process according to claim 14.

22. The process according to claim 21, wherein a separate trioxane synthesis stage is carried out and precedes the first distillation stage.

23. The process according to claim 21, wherein the trioxane synthesis stage and the first distillation stage are carried out together as a reactive distillation.

24. The process according to claim 21, wherein the stream X contains from 55 to 85% by weight of formaldehyde and from 15 to 45% by weight of water.

25. The process according to claim 21, wherein the stream X is obtained from an aqueous formaldehyde solution of low formaldehyde concentration by concentrating in an evaporator.
